(19) United States
(12) Patent Application Publication
(10) Pub. No.: US 2006/0235378 A1
(43) Pub. Date: Oct. 19, 2006

(54) Slider control for ablation handset

(75) Inventor: Luke Waaler, Fort Collins, CO (US)

Correspondence Address: UNITED STATES SURGICAL,;A DIVISION OF TYCO HEALTHCARE GROUP LP, 195 MCDERMOTT ROAD, NORTH HAVEN, CT, 06473, US

(73) Assignee: Sherwood Services AG

(21) Appl. No.: 11/109,196

(22) Filed: Apr. 18, 2005

(51) Int. Cl. A61B 18/14

(52) U.S. Cl. 606/41; 606/42

(57) ABSTRACT

An electrosurgical ablation instrument connectable to an electrosurgical energy source is provided. The ablation instrument includes a handle; at least one elongate probe electrode extending from an end of the handle, each probe electrode including at least a conductive distal tip; and an intensity controller operatively supported on the handle. The intensity controller adjusts the level of energy delivered from the electrosurgical energy source to the conductive distal tips of each probe electrode.

### BACKGROUND

1. Technical Field

The present disclosure relates generally to electrosurgical instruments and, more particularly, to radiofrequency ablation assemblies having hand accessible variable controls.

2. Background of Related Art

The use of radiofrequency/electrosurgical electrodes and/or probes for the ablation of tissue in a patient's body is known. In a typical situation, an electrosurgical electrode comprising an elongated, cylindrical shaft, with a portion of its external surface insulated, is inserted into the patient's body. The electrode typically has an exposed conductive tip, which is used to contact body tissue in the region where the heat lesion or ablation is desired. The electrode is connected to an electrosurgical power source, such as a generator, which provides radiofrequency voltage to the electrode, and which, in turn, transmits the radiofrequency current into the tissue near its exposed conductive tip. This radiofrequency current usually returns to the electrosurgical power source through a reference electrode, e.g., a return electrode, which may comprise a large area conductive contact, connected to an external portion of the patient's body. This configuration has been described in articles, as for example, a research paper by Cosman, et al., entitled “Theoretical Aspects of Radiofrequency Lesions in the Dorsal Root Entry Zone,” Neurosurgery, December 1984, Vol. 15, No. 6, pp 945-950, and a research paper by Goldberg, et al. entitled “Tissue Ablation with Radiofrequency: Effective Probe Size, Gauge, Duration, and Temperature and Lesion Volume” Acad Radio., 1995, Vol. 2, No. 5, pp 399-404. Radiofrequency lesion generators and electrode systems such as those described above are commercially available from Valleylab, Inc. a division of Tyco Healthcare LP, located in Boulder, Colo.

To enlarge ablation volumes, electrodes with curved conductive tips have been proposed. Such tips are injected from a cylindrical electrode placed near the targeted or desired tissue volume to produce an off-axis, curved arc within the targeted or desired tissue. In this way, off-axis ablation volumes may be produced away from the central axis of the inserted cannula. The off-axis lesions produced by these off-axis radiofrequency electrodes enlarge the lesion volume away from an axially symmetric, exposed electrode tip. One example of this type of an off-axis electrode is the Zervas Hypophysectomy Electrode available from the company Radionics, Inc., located in Burlington, Mass. Another example of this type of an off-axis electrode is the multiple side-emitting, off-axis electrode made by Radiotherapeutics, located in Mountainview, Calif. The multiple electrode elements range in curved arcs at various azimuthal angles. By making an umbrella of off-axis tip extensions at various azimuthal angles relative to a central insertion cannula, an enlarged lesion volume can be produced. Disadvantages of irregular heat ablation shapes and large central cannula sizes are discussed below.

Also, pairs of electrodes have been inserted into the body in a bipolar configuration, typically in parallel pairs held close to each other. Examples of such bipolar configurations are available from the company Elekta AB, located in Stockholm, Sweden. In such bipolar configurations, one electrode may serve as a source and the other may serve as a sink for the radiofrequency current from the RF generator. In other words, one electrode is disposed at the opposite voltage (pole) to the other so that current from the radiofrequency generator is drawn directly from one electrode to the other. The primary purpose of a bipolar electrode arrangement is to insure more localized and smaller heat ablation volumes. With such configurations, the ablation volume is restricted to the region between the bipolar electrodes.

Electrodes with cooled conductive tips have been proposed by Goldberg, et al., in their article referenced above. With cooling, electrode tips generally produce larger lesion volumes as compared with radiofrequency electrodes, which are not cooled.

Hyperthermia is a method of heating tissue, which contains a cancerous tumor, to thermally non-lethal levels, typically less than 45 degrees Centigrade, combined with irradiation of the tissue with X-rays. Such application of mild non-lethal heating in combination with radiation by X-rays enhances destruction of cancer cells while sparing the normal cells from being killed. For hyperthermia, multiple arrays of high frequency electrodes are implanted in tumors. The electrodes are typically placed in a dispersed fashion throughout the tumor volume to cover the tumor volume with uniform heat, which is below the lethal 45 degree level. The electrodes are sequentially applied with high frequency voltage so that each electrode heats in sequence its neighborhood tissue and then shuts off. Then, the next electrode does the same in a time series. This sequence of cycling the voltage through the electrodes continues at a prescribed frequency and for a time period ranging anywhere from minutes to hours. The primary objective of hyperthermia is not to fully ablate tumors by outright heat destruction of the cancerous tumor. On the contrary, its objective is to avoid temperatures above 45 degrees C. anywhere in the treatment volume. The article by Melvin A. Astrahan entitled “A Localized Current Field Hyperthermia System for Use with 192-Iridium Interstitial Implants,” in Medical Physics, 9(3), May/June 1982, describes the technique of radiofrequency hyperthermia.

The electrode systems discussed above typically produce various sized lesion volumes. For example, standard single cylindrical electrodes, with cool tips as described above, produce lesion volumes up to about 3 to 4 cm in diameter in living tissue, such as the liver, using cannulae of about 1 to 2 mm in diameter and an exposed tip length of about several centimeters. The umbrella lesions made by multiple side-emerging, exposed tips, also produce lesion volumes of about 3 to 4 cm in diameter.

Typically, during an ablation procedure, the surgeon must adjust the power intensity delivered from the electrosurgical generator to the exposed conductive tip of the electrode(s). This often entails either rotation of a dial or movement of a slide located on the electrosurgical generator. In order to do so, the surgeon must extend his hand from the operating field (i.e., typically considered a sterile field and/or environment) and touch, adjust and/or manipulate the controls of the electrosurgical generator which is outside of the operating field (i.e., typically considered a non-sterile field and/or environment). Alternatively, the surgeon must ask another individual (e.g., an assistant, a technician or the like) to adjust the controls and/or power level of the electrosurgical generator so that the surgeon's hand does not contact an object out side of the operating field and become contaminated.

A need exists for a system and/or method for controlling the power intensity delivered to the exposed conductive tip of the electrode while in the sterile field, without having to touch objects in the non-sterile field.

A need also exists for a system and/or method for controlling the power intensity delivered to the exposed conductive tip of the electrode directly from the ablation assembly.

### SUMMARY

The present disclosure is directed to electrosurgical instruments having variable controls.

According to an aspect of the present disclosure, an electrosurgical ablation instrument connectable to an electrosurgical energy source is provided. The ablation instrument includes a handle; at least one elongate probe electrode extending from an end of the handle, each probe electrode including at least a conductive distal tip; and an intensity controller operatively supported on the handle. The intensity controller adjusts the level of energy delivered from the electrosurgical energy source to the conductive distal tips of each probe electrode.

In an embodiment, the intensity controller is a slide button slidably supported on the handle. Accordingly, in use, when the slide is positioned at a distal-most location a maximum level of energy is delivered from the electrosurgical energy source to the conductive distal tips of each probe electrode. Additionally, when the slide button is positioned at a proximal-most location a minimum level of energy is delivered from the electrosurgical energy source to the conductive distal tips of each probe electrode. The slide button may be positioned at a proximal-most location no energy is delivered from the electrosurgical energy source to the conductive distal tips of each probe electrode.

It is envisioned that the ablation instrument further includes an activation button operatively supported on the handle.

Each probe electrode is desirably electrically conductive along its entire length and includes an insulative material covering at least a portion of the length thereof to expose at least the distal tip thereof. Each probe electrode may be fluidly cooled.

The ablation instrument may include a plug assembly for selective operative connection to a complementary receptacle provided on the electrosurgical energy source; and a connecting wire interconnecting the plug assembly to each electrode probe. Three elongate electrode probes may be included which extend from the handle.

In another embodiment, it is envisioned that the intensity controller is a dial rotatably supported on the handle.

According to another aspect of the present disclosure, an electrode array system is provided. The electrode array system includes an electrosurgical energy source; and an electrosurgical ablation instrument connectable to an electrosurgical energy source. The ablation instrument includes a handle; at least one elongate probe electrode extending from an end of the handle, each probe electrode including at least a conductive distal tip; and an intensity controller operatively supported on the handle. The intensity controller adjusts the level of energy delivered from the electrosurgical energy source to the conductive distal tips of each probe electrode.

It is envisioned that the intensity controller of the ablation instrument is a slide button slidably supported on the handle. Accordingly, in use, when the slide button is positioned at a distal-most location a maximum level of energy is delivered from the electrosurgical energy source to the conductive distal tips of each probe electrode. Additionally, when the slide button is positioned at a proximal-most location a minimum level of energy is delivered from the electrosurgical energy source to the conductive distal tips of each probe electrode. In an embodiment, when the slide button is positioned at a proximal-most location no energy is delivered from the electrosurgical energy source to the conductive distal tips of each probe electrode.

The ablation instrument may further include an activation button operatively supported on the handle.

Each probe electrode of the ablation instrument may be electrically conductive along its entire length and includes an insulative material covering at least a portion of the length thereof to expose at least the distal tip thereof. It is contemplated that each probe electrode of the ablation instrument is fluidly cooled.

The ablation instrument may further include a plug assembly for selective operative connection to a complementary receptacle provided on the electrosurgical energy source; and a connecting wire interconnecting the plug assembly to each electrode probe.

In an embodiment, three elongate electrode probes extend from the handle.

In an alternate embodiment, the intensity controller of the ablation instrument is a dial rotatably supported on the handle.

These and other objects will be more clearly illustrated below by the description of the drawings and the detailed description of the preferred embodiments.

### BRIEF DESCRIPTION OF THE DRAWINGS

The accompanying drawings, which are incorporated in and constitute a part of this specification, illustrate embodiments of the invention, and together with a general description of the invention given above, and the detailed description of the embodiments given below, serve to explain the principles of the invention.

FIG. 1 is a schematic illustration of an ablation electrode array system according to the present disclosure showing multiple radiofrequency electrodes being positioned into a patient's organ for producing heat ablation of a targeted tissue area;

FIG. 2 is a further schematic illustration of the ablation electrode array system of the present disclosure; and

FIG. 3 is an enlarged, perspective view of a portion of an ablation instrument in accordance with another embodiment of the present disclosure.

### DETAILED DESCRIPTION OF EMBODIMENTS

Particular embodiments of the presently disclosed ablation electrode array system will now be described in detail with reference to the drawing figures wherein like reference numerals identify similar or identical elements. As used herein, the term “distal” refers to that portion which is further from the user while the term “proximal” refers to that portion which is closer to the user or surgeon.

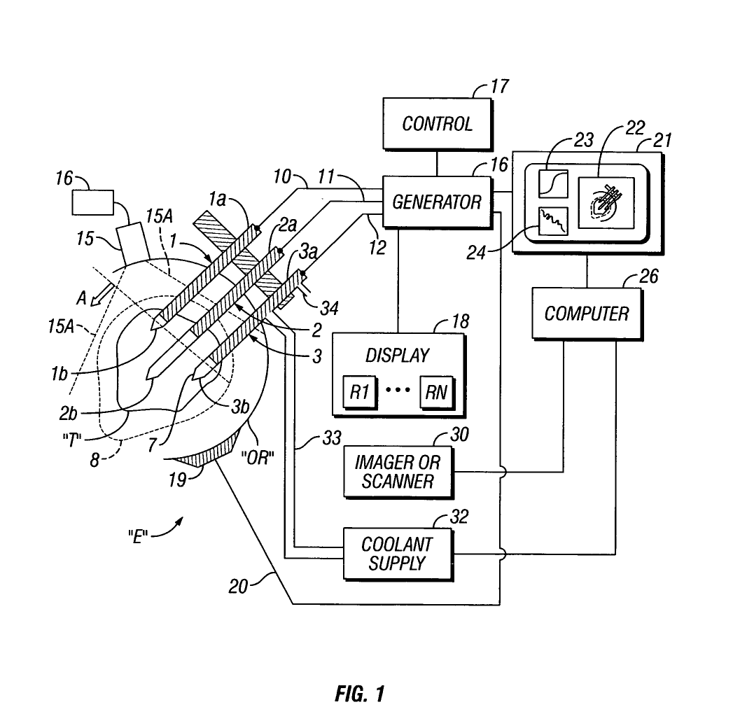

Referring initially to FIG. 1, an embodiment of a multiple electrode arrangement such as an ablation electrode array system, in accordance with an embodiment of the present disclosure, is generally designated “E”. Electrode array system “E” includes a plurality of elongate probe electrodes 1, 2 and 3, which are to be inserted into an organ “OR” of a human body or any other body tissue. Respective distal tips 1b, 2b and 3b of electrodes 1, 2 and 3 are uninsulated and conductively exposed so that electrical currents induce heating within the tissue or organ “OR”. A targeted volume of tissue “T” is shown in sectional view and may represent, for example, a tumor or other abnormality in a human body.

Electrodes 1, 2 and 3 are connected by respective wires or cables 10, 11 and 12 to an electrosurgical energy source, such as, for example, an electrosurgical generator 16. Electrosurgical generator 16 may be a radiofrequency or high frequency type generator. Electrosurgical generator 16 may include control elements, illustrated by block 17, which may, for example, increase the radiofrequency power output of electrodes 1, 2 and 3, control temperature when electrode array system “E” or satellite sensors (not shown) include temperature sensors, monitor or control impedance, power, current, voltage, or other output parameters. In an alternate embodiment, as will be described in greater detail below, it is envisioned that electrosurgical generator 16 may be free of control elements and the like.

Electrosurgical generator 16 may include a display or screen, illustrated by block 18, within it or as a separate system, for providing a display of heating parameters such as temperature for one or more of electrodes 1, 2 and 3, impedance, power, current, or voltage of the radiofrequency output. Such individual display readings are illustrated by the reference letters “R1 . . . RN”. It is further envisioned that display or screen 18 may be a touch screen or the like which is responsive to touches by a surgeon, technician, or the like.

Electrode system “E” further includes a reference electrode 19, which may be placed in contact with the skin of a patient or an external surface of organ “OR” with a connection 20 to electrosurgical generator 16. Reference electrode 19 and connection 20 serves as a path for return current from electrosurgical generator 16 through electrodes 1, 2 and 3.

Each electrode 1, 2 and 3 includes a rigid shaft 1a, 2a and 3a, respectively, which enables electrodes 1, 2 and 3 to be easily urged into the body tissue or organ “OR”. Each electrode 1, 2 and 3 terminates pointed distal tips 1b, 2b and 3b, respectively. A portion of the external surface of each electrode 1, 2 and 3 may be covered with an insulating material, as indicated by hatched line areas in FIG. 1. Distal tips 1b, 2b and 3b are connected, through respective shafts 1a, 2a and 3a to cables 10, 11 and 12, respectively, and thereby to electrosurgical generator 16.

By way of example only and in no way to be considered as limiting, electrosurgical generator 16 may be a radiofrequency generator with frequency between about 100 kilohertz (kHz) to several hundred megahertz (MHz). Additionally, electrosurgical generator 16 may have power output ranging from several watts to several hundred watts, depending on the clinical application.

Electrodes 1, 2 and 3 may be raised to the same radiofrequency voltage potential from electrosurgical generator 16. The array of electrodes thus becomes, in effect, a larger, coherent electrode including the individual electrode tips 1b, 2b and 3b. Thus, the heating effect of the array of electrodes is substantially similar to that achieved by one large single electrode.

As seen in FIG. 1, by way of illustration only, a targeted region to be ablated is represented in sectional view by the line “T”. It is desired to ablate the targeted region “T” by fully engulfing targeted region “T” in a volume of lethal heat elevation. The targeted region “T” may be, for example, a tumor which has been detected by an image scanner 30. For example, CT, MRI, or ultrasonic image scanners may be used, and the image data transferred to a computer 26. As an alternate example, an ultrasonic scanner head 15 may be disposed in contact with organ “OR” to provide an image illustrated by lines 15A. A data processor 16 may be connected to the display devices to visualize targeted region “T” and/or ablation zone “T1” in real time during the ablation procedure.

The image representation of the scan may be displayed on display unit 22 to represent the size and position of target region “T”. Placement of electrodes 1, 2 and 3 may be predetermined based on such image data as interactively determined by real-time scanning of organ “OR”. Electrodes 1, 2 and 3 are inserted into the tissue by freehand technique by a guide block or introducer 100 with multi-hole templates, or by stereotactic frame or frameless guidance, as known by those skilled in the art.

An array of electrodes 1, 2 and 3 may be connected to the same radiofrequency voltage from electrosurgical generator 16. Accordingly, the array of electrodes 1, 2 and 3 will act as a single effectively larger electrode. The relative position and orientation of electrodes 1, 2 and 3 enable the creation of different shapes and sizes of ablation volumes. For example, in FIG. 1, dashed line 8 represents the ablation isotherm in a sectional view through organ “OR”. Such an ablation isotherm may be that of the surface achieving possible temperatures of approximately 50° C. or greater. At that temperature range, sustained for approximately 30 seconds to approximately several minutes, tissue cells will be ablated. The shape and size of the ablation volume, as illustrated by dashed line 8, may accordingly be controlled by the configuration of the electrode array, the geometry of the distal tips 1b, 2b and 3b of electrodes 1, 2 and 3, respectively, the amount of RF power applied, the time duration that the power is applied, cooling of the electrodes, etc.

As seen in FIG. 1, optionally each electrode 1, 2 and 3 of electrode array system “E” may be fluidly connected to a coolant supply system 32 or the like via conduits 33. Coolant supply system 32 delivers fluid to each electrode 1, 2 and 3 to thereby cool distal tips 1b, 2b and 3b and enable enlargement of ablation volume 8.

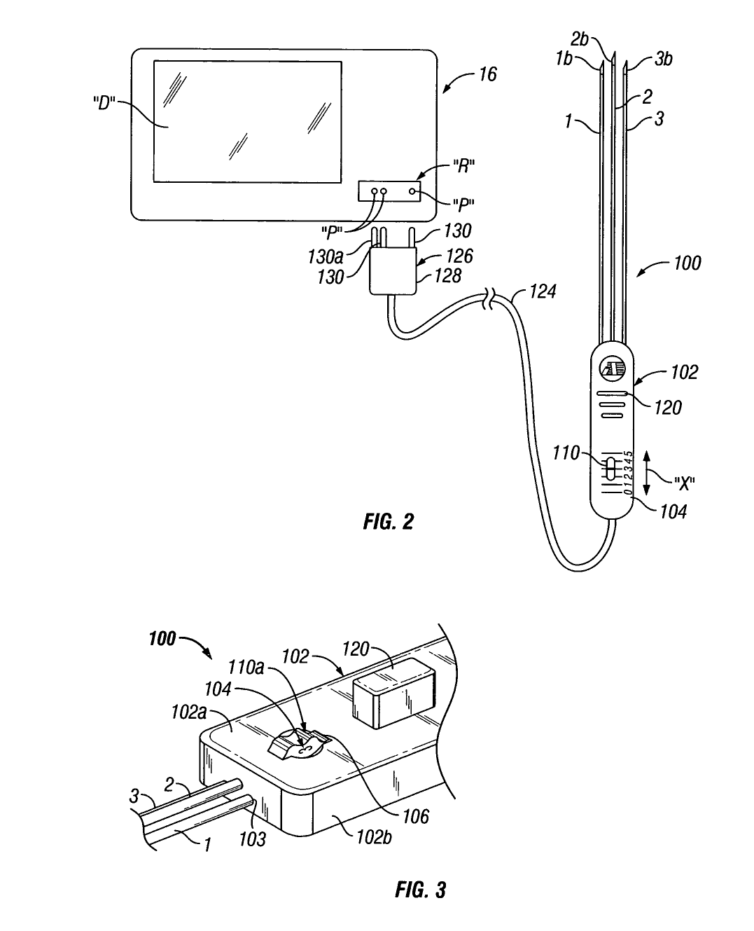

As seen in FIGS. 2 and 3, electrode array system “E” includes an electrosurgical ablation instrument 100 which is electrically connectable to electrosurgical generator 16. As seen in FIG. 3, ablation instrument 100 includes a housing 102, which may have a top-half shell portion 102a and a bottom-half shell portion 102b. Housing 102 may include distal openings 103 through which electrodes 1, 2 and 3 extend, and a proximal opening (not shown), through which connecting wire 124 extends. Top-half shell portion 102a and bottom-half shell portion 102b may be secured and/or bonded together using methods known by those skilled in the art.

As seen in FIG. 2, ablation instrument 100 may be coupled to electrosurgical generator 16 via a plug assembly 126 operatively connected to an end of connecting wire 124. Plug assembly 126 includes a housing portion 128 having a first half-section and a second half-section operatively engageable with one another (not shown), preferably, via a snap-fit engagement. Plug assembly 126 includes a power pin 130 extending distally from housing portion 128. Preferably, power pin 130 is positioned to be off center, i.e., closer to one side edge of housing portion 128 than the other. Plug assembly 126 further includes at least one, preferably, a pair of position pins 132a, 132b also extending from housing portion 128. Position pins 132a, 132b are oriented in the same direction as power pin 130. A first position pin 132a may be positioned to be off center and in close proximity to an opposite side edge of housing portion 128 as compared to power pin 130 and a second position pin 132b is positioned in close proximity to a center of housing portion 128. Pins 130, 132a and 132b of plug 126 are preferably disposed on housing portion 128 at locations which correspond to pin receiving positions “P” of a connector receptacle “R” of electrosurgical generator 16.

The location of pins 130, 132a and 132b functions as a polarization member, ensuring that power pin 130 is properly received in connector receptacle “R” of electrosurgical generator 16.

Ablation instrument 100 may include at least one activation button 120 extending through housing 102. Each activation button 120 desirably controls the transmission of RF energy supplied from electrosurgical generator 16 to distal tips 1b, 2b and 3b of electrodes 1, 2 and 3, respectively.

It is contemplated that ablation instrument 100 includes an intensity controller in the form of a slide button 110 slidably supported on housing 102. Slide button 110 is in operative engagement with a potentiometer (not shown), operatively supported within housing 102, for adjusting the RF power and/or intensity level of energy delivered from electrosurgical generator 16 to distal tips 1b, 2b and 3b of electrodes 1, 2, and 3, respectively. The potentiometer may be a film-type potentiometer.

In use, as slide button 110 is moved or slid along housing 102, the intensity of the RF energy delivered to distal tips 1b, 2b and 3b of electrodes 1, 2 and 3, respectively, is varied. For example, when slide 110 is positioned at a proximal-most location a minimum level of or amount of RF energy or no RF energy/power is transmitted to distal tips 1b, 2b and 3b of electrodes 1, 2 and 3, respectively. Additionally, when slide button 110 is positioned at a distal-most location a maximum level of or amount of RF energy is transmitted to distal tips 1b, 2b and 3b of electrodes 1, 2 and 3, respectively. As can be appreciated, the minimum amount of RF energy may be transmitted when slide button 110 is positioned at a distal-most location, and the maximum amount of RF energy may be transmitted when slide button 110 is at a proximal-most location.

Slide button 110 is configured and adapted to adjust the energy or power parameters (e.g., voltage, power and/or current intensity) and/or the power verses impedance curve shape to affect the perceived output intensity. For example, the greater slide button 110 is displaced in a distal direction the greater the level of the power parameters transmitted to distal tips 1b, 2b and 3b of electrodes 1, 2 and 3, respectively. Alternatively, it is envisioned that slide button 110 may be displaced proximally to increase the power parameters.

The intensity settings are preferably preset and selected from a look-up table based on a desired surgical effect, surgical specialty and/or surgeon preference. The selection may be made automatically or selected manually by the user. The intensity values may be predetermined or adjusted by the user.

In operation, the surgeon activates ablation instrument 100 by either depressing activation button 120 or by manipulating some other form of switch or the like (e.g., a foot switch) thereby transmitting RF energy from electrosurgical generator 16 to distal tips 1b, 2b and 3b of electrodes 1, 2 and 3, respectively. In order to vary the intensity of the RF energy delivered, the surgeon displaces slide button 110, in a direction indicated by double-headed arrow “X” (see FIG. 2). The intensity of RF energy delivered may be varied from approximately 60 mA for a light effect to approximately 240 mA for a more aggressive effect. For example, by positioning slide button 110 closer to the proximal-most end of housing 102 a lower intensity level is produced, and by positioning slide button 110 closer to the distal-most end of housing 102 a larger intensity level is produced. It is envisioned that when slide button 110 is positioned at the proximal-most end of housing 102, the potentiometer is set to a null and/or open position.

It is contemplated that slide button 110 and housing 102 may be provided with tactile feedback elements in the form of a series of cooperating discreet or detented positions defining a series of positions, preferably five, to allow easy selection of the output intensity from the low intensity setting to the high intensity setting. The series of cooperating discreet or detented positions also provide the surgeon with a degree of tactile feedback. Accordingly, in use, as slide button 110 is moved distally or proximally along housing 102 the tactile feedback elements provide the user with tactile indications as to when the intensity controller has been set to the desired intensity setting and RF energy setting. Alternatively, audible feedback can be produced from electrosurgical generator 16 (e.g., a “tone”) and/or from an auxiliary sound-producing device such as a buzzer (not shown).

As seen in FIG. 2, housing 102 includes a series of indicia 104 provided thereon which are visible to the user. Indicia 104 may be a series of numbers (e.g., numbers 1-5) which reflect the level of intensity that is to be transmitted. Indicia 104 may be provided alongside slide button 110. Indicia 104 is preferably provided on housing 102 and spaced therealong to correspond substantially with the location of the tactile feedback elements. Accordingly, as slide button 110 is moved distally and proximally, slide button 110 comes into registration with particular indicia 104 which corresponds to the location of the tactile feedback elements. For example, indicia 104 may include numeric characters (as shown in FIG. 2), alphabetic character, alphanumeric characters, graduated symbols, graduated shapes, and the like.

With continued reference to FIG. 2, electrosurgical generator 16 includes a touch screen display “D”. All electrosurgical functions may be controlled through touch screen display “D” of electrosurgical generator 16. However, in accordance with the present disclosure, the level of energy delivered to distal tips 1b, 2b and 3b of electrodes 1, 2 and 3, respectively, may be controlled from ablation instrument 100. Accordingly, this reduces the need for the surgeon to make contact with an object outside of the sterile field, during the surgical procedure, in order to adjust the energy levels.

Handle 102 may be ergonomic and may include soft-touch material provided thereon in order to increase the comfort, gripping and manipulation of ablation instrument 100.

While the intensity controller has been shown and described as a slide button 110, as seen in FIG. 3, it is envisioned and within the scope of the present disclosure for the intensity controller to be a dial or knob 110a rotatably supported on or at least partially within an aperture 106 of housing 102. Dial 110a may be positioned distally or forward of activation button 120 such that dial 110a is not inadvertently rotated during the depression of activation button 120. As seen in FIG. 3, a surface of dial 110a may be provided with indicia and/or markings 104 in the form of alphanumeric characters and the like to indicate to the surgeon the degree of and/or level of energy at which ablation instrument 100 is set. Accordingly, in use, as dial 110a is rotated the level of RF energy delivered to distal tips 1b, 2b and 3b of electrodes 1, 2 and 3, respectively, is adjusted.

It is also envisioned that ablation instrument 100 may include a smart recognition technology which communicates with the generator to identify the ablation instrument and communicate various surgical parameters which relate to treating tissue with ablation instrument 100. For example, ablation instrument 100 may be equipped with a bar code or Aztec code which is readable by electrosurgical generator 16 and which presets electrosurgical generator 16 to default parameters associated with treating tissue with ablation instrument 100. The bar code or Aztec code may also include programmable data which is readable by electrosurgical generator 16 and which programs electrosurgical generator 16 to specific electrical parameters prior to use.

Other smart recognition technology is also envisioned which enable electrosurgical generator 16 to determine the type of instrument being utilized or to insure proper attachment of the instrument to the generator as a safety mechanism. One such safety connector is identified in U.S. patent application Ser. No. 10/718,114, filed Nov. 20, 2003, the entire contents of which being incorporated by reference herein. For example, in addition to the smart recognition technology described above, such a safety connector can include a plug or male portion operatively associated with ablation instrument 100 and a complementary socket or female portion operatively associated with electrosurgical generator 16. Socket portion is “backward compatible” to receive connector portions of ablation instruments 100 disclosed therein and to receive connector portions of prior art electrosurgical instruments.

Although the subject apparatus has been described with respect to preferred embodiments, it will be readily apparent, to those having ordinary skill in the art to which it appertains, that changes and modifications may be made thereto without departing from the spirit or scope of the subject apparatus.

What is claimed is:

1. An electrosurgical ablation instrument connectable to an electrosurgical energy source, the ablation instrument comprising:
a handle;
at least one elongate probe electrode extending from an end of the handle, each probe electrode including at least a conductive distal tip; and
an intensity controller operatively supported on the handle, wherein the intensity controller adjusts the level of energy delivered from the electrosurgical energy source to the conductive distal tips of each probe electrode.

2. The ablation instrument according to claim 1, wherein the intensity controller is a slide button slidably supported on the handle.

3. The ablation instrument according to claim 2, wherein when the slide button is positioned at a distal-most location a maximum level of energy is delivered from the electrosurgical energy source to the conductive distal tips of each probe electrode, and when the slide button is positioned at a proximal-most location a minimum level of energy is delivered from the electrosurgical energy source to the conductive distal tips of each probe electrode.

4. The ablation instrument according to claim 3, wherein when the slide button is positioned at a proximal-most location no energy is delivered from the electrosurgical energy source to the conductive distal tips of each probe electrode.

5. The ablation instrument according to claim 4, further comprising an activation button operatively supported on the handle.

6. The ablation instrument according to claim 5, wherein each probe electrode is electrically conductive along its entire length and includes an insulative material covering at least a portion of the length thereof to expose at least the distal tip thereof.

7. The ablation instrument according to claim 6, wherein each probe electrode is fluidly cooled.

8. The ablation instrument according to claim 5, further comprising:
a plug assembly for selective operative connection to a complementary receptacle provided on the electrosurgical energy source; and
a connecting wire interconnecting the plug assembly to each electrode probe.

9. The ablation instrument according to claim 8, wherein three elongate electrode probes extend from the handle.

10. The ablation instrument according to claim 1, wherein the intensity controller is a dial rotatably supported on the handle.

11. An electrode array system, comprising:
an electrosurgical energy source; and
an electrosurgical ablation instrument connectable to an electrosurgical energy source, the ablation instrument including:
a handle;
at least one elongate probe electrode extending from an end of the handle, each probe electrode including at least a conductive distal tip; and
an intensity controller operatively supported on the handle, wherein the intensity controller adjusts the level of energy delivered from the electrosurgical energy source to the conductive distal tips of each probe electrode.

12. The electrode array system according to claim 11, wherein the intensity controller of the ablation instrument is a slide button slidably supported on the handle.

13. The electrode array system according to claim 12, wherein when the slide button of the ablation instrument is positioned at a distal-most location a maximum level of energy is delivered from the electrosurgical energy source to the conductive distal tips of each probe electrode, and when the slide button of the ablation instrument is positioned at a proximal-most location a minimum level of energy is delivered from the electrosurgical energy source to the conductive distal tips of each probe electrode.

14. The electrode array system according to claim 13, wherein when the slide button of the ablation instrument is positioned at a proximal-most location no energy is delivered from the electrosurgical energy source to the conductive distal tips of each probe electrode.

15. The electrode array system according to claim 14, wherein the ablation instrument further comprises an activation button operatively supported on the handle.

16. The electrode array system according to claim 15, wherein each probe electrode of the ablation instrument is electrically conductive along its entire length and includes an insulative material covering at least a portion of the length thereof to expose at least the distal tip thereof.

17. The electrode array system according to claim 16, wherein each probe electrode of the ablation instrument is fluidly cooled.

18. The electrode array system according to claim 15, wherein the ablation instrument further comprises:
a plug assembly for selective operative connection to a complementary receptacle provided on the electrosurgical energy source; and
a connecting wire interconnecting the plug assembly to each electrode probe.

19. The electrode array system according to claim 18, wherein three elongate electrode probes extend from the handle.

20. The electrode array system according to claim 11, wherein the intensity controller of the ablation instrument is a dial rotatably supported on the handle.
